(19) United States
(12) Patent Application Publication
(10) Pub. No.: US 2017/0360766 A1
(43) Pub. Date: Dec. 21, 2017

(54) 3-(PIPERIDIN-4-YL)-ISOXAZOL-3(OH)-ONES FOR TREATMENT OF DERMATOLOGIC DISORDERS

(71) Applicant: Emeriti Pharma AB, Mölndal (SE)

(72) Inventors: Tomas Fex, Västra Frölunda (SE); Nils David Gustafsson, Kullavik (SE)

(73) Assignee: Emeriti Pharma AB, Mölndal (SE)

(21) Appl. No.: 15/536,288

(22) PCT Filed: Dec. 18, 2015
(86) PCT No.: PCT/SE2015/051369

§ 371 Date: Jun. 15, 2017

Related U.S. Application Data
(60) Provisional application No. 62/094,278, filed on Dec. 19, 2014.

(51) Int. Cl. A61K 31/454; A61Q 1/02

(52) U.S. Cl. CPC A61K 31/454; A61Q 1/02; A61K 2300/00

(57) ABSTRACT

Described are a group of isoxazol-3(2H)-one analogues and their use in topical formulations for the treatment and prophylaxis of dermatological disorders.

### FIELD OF THE INVENTION

The present invention relates to compounds for use in the treatment of dermatological disorders. More specifically, the invention relates to certain 5-(piperidin-4-yl) isoxazol-3(2H)-one derivatives for use in the treatment of dermatological disorders.

### BACKGROUND

In the patent application WO2010117323 (A1) are described a group of isoxazol-3(2H)-one analogues and their use in treating fibrinolysis related diseases or conditions, for example inherited bleeding disorders, stroke, menorrhagia and liver diseases and for the treatment of hereditary angioedema (a systemic disorder usually caused by C1-esterase deficiency (Longhurst 2012)). Thus, in the patent application WO2010117323 (A1) dermatological disorders are not contemplated.

The mode of action of epsilon amino-caproic acid (EACA), tranexamic acid (TXA) and compounds of the patent application WO2010117323 (A1) are inhibition of the binding of plasminogen to fibrin in the blood clot. Upon binding to fibrin plasminogen undergoes a conformational change whereby it can be activated by plasminogen activators, tPA and uPA, to plasmin which can degrade fibrin to fibrin degradation products in the blood clot. Plasminogen is initially binding to fibrin C-terminal lysine residues via the protein structure kringle 1. EACA and TXA and compounds of the patent application WO2010117323 (A1) are binding to kringle 1 of plasminogen. EACA and TXA were originally developed as lysine analogues (Dunn 1999).

The clinical effects of EACA and TXA are mainly related to reducing bleeding due to inhibition of fibrinolysis and hence EACA and TXA are popularly called fibrinolysis inhibitors. However, plasminogen not only binds to fibrin but to many other biological surfaces and has many other substrates than fibrin. Thus, plasminogen can bind to other fibrillar proteins like laminin and plasminogen receptors on cells and cell-derived microparticles. Furthermore, plasmin generated on these surfaces have effects on numerous targets leading to e.g. facilitated cell migration, chemotaxis and proinflammatory cell activation (for references and details see Syrovets 2012, and Plow 2012). As a consequence excessive activation of plasmin in chronic inflammatory or autoimmune diseases may exacerbate the activation of inflammatory cells and the pathogenesis of the disease (Syrovets 2012). The plasminogen receptors and plasminogen/plasmin are also considered important for the growth and spread of tumour cells as documented in experimental studies (see reviews by Madureira 2012 and Ceruti 2013).

The literature on TXA in dermatology describe mainly the use of tranexamic acid in the treatment of melasma, where oral use may be effective while the effect of topical administration seem more questionable (see review by Tse 2013). Other dermatological indications are not much studied by TXA and EACA.

However, the role of plasminogen and plasmin are considered in other dermatological disorders. Examples of such disorders are atopic dermatitis, psoriasis, and rosacea.

In atopic dermatitis there is an increased permeability of stratum corneum, the outermost layer of the skin, which leads to an increased loss of water and a dry skin sensation. The increased permeability also leads to a susceptibility to inflammation. The proteases in stratum corneum in dry skin conditions have recently been reviewed (Rawlings 2013). Although focus of the review is on kallikreins, important for normal skin desquamation, plasminogen is found in stratum corneum and plasminogen can be activated by kalikreins to plasmin, and plasmin in turn can activate prekallikreins to kallikreins. The plasminogen system in the epidermis is thought to be one of the major protease activities involved in the delay of stratum corneum barrier recovery after barrier damage (see Rawlings 2013). Thus, it has been shown that topically applied TXA can enhance stratum corneum barrier recovery in healthy volunteer after an experimental damage to stratum corneum (Kitamura 1995, Denda 1997, Yuan 2014).

Psoriatic lesions display increased expression of plasminogen or increased plasmin activity (Jensen 1988, Jensen 1990, Spiers 1994). Another study found that plasminogen levels were diminished, most of it having been transformed into active plasmin. Also, levels of annexin II, a receptor for activation of plasminogen to plasmin, were increased in both dermis and epidermis in psoriasis. Plasmin at sites of inflammation was pro-inflammatory (Li 2011).

In rosacea, there seem to be a decreased stratum corneum barrier function at lesional sites. We are aware of three clinical studies evaluating either TXA or EACA in patients with rosacea. Wu 2010 (meeting abstract in English), Sun 2013 (paper in Chinese) and Zhong 2015 (paper in English) are the same study. In addition, there is one study from South Korea (Kim 2013) and one form the USA (Two 2013). In one study topical TXA improved stratum corneum barrier function (Wu 2010, Sun 2013, Zhong 2015) and in the study with EACA a reduction in stratum corneum protease activity (Two 2014) was observed. The authors of all three studies indicated a trend towards clinical improvement (Wu 2010, Sun 2013 and Zhong 2015, Kim 2012, Two 2014). The studies used high concentrations of drug in the formulations, 3% (˜0.2M) TXA (Wu 2010, Sun 2013 and Zhong 2015), 10% (˜0.6M) TXA for soaking in wet gauzes for 20 min (Kim 2012) and ˜13% EACA (1 M) cream (Two 2014).

The two proteins uPA and PAI-1, both involved in the activation of plasminogen to plasmin, are among the best validated prognostic biomarkers currently available for lymph node-negative breast cancer (Duffy 2014) indicating that the fibrinolytic pathway may be involved in cancer formation, growth and spread in patients.

Side effects can be associated with TXA. Oral TXA is reported to give allergic skin reaction in <1/100 treated (CYCLO-f, EMA 2000). In a double blind, randomized, prospective study, topical (5% (˜0.3M) TXA) versus vehicle had no effect on melasma but topical TXA produced erythema (Ayuthaya 2012).

### SUMMARY OF THE INVENTION

One aspect relates to 5-(piperidin-4-yl) isoxazol-3(2H)-one derivatives, including pharmaceutically suitable salts, hydrates, solvates and prodrugs thereof, for use in the treatment of a dermatological disorder.

Compounds of the invention have higher affinity for plasminogen kringle 1 than TXA and EACA, thus potentially providing improved treatment and prophylaxis of dermatological disorders.

Being more potent and more lipophilic than TXA and EACA they are more likely to reach effective concentrations in the skin, and have also a likelihood of developing fewer local side effects.

The compounds are formulated for topical administration to be used in a method of treatment of dermatological disorders.

### BRIEF DESCRIPTION OF THE DRAWINGS

FIG. 1 is a graph showing the cumulative amount on the receiver side of the pig skin membrane as a function of time, of 0.5% 5-[(2R,4S)-2-(2,2-dimethylpropyl)piperidin-4-yl]-2,3-dihydro-1,2-oxazol-3-one, dissolved in phosphate buffered saline (PBS), with and without propylene glycol (PG).

FIG. 2 is a graph showing the steady state flux through the pig skin membrane of 0.5% of 5-[(2R,4S)-2-(2,2-dimethylpropyl)piperidin-4-yl]-2,3-dihydro-1,2-oxazol-3-one, dissolved in phosphate buffered saline (PBS), with and without propylene glycol (PG).

FIG. 3 is a graph showing the steady state flux through the pig skin membrane of various concentrations of 5-[(2R,4S)-2-(2,2-dimethylpropyl)piperidin-4-yl]-2,3-dihydro-1,2-oxazol-3-one dissolved in Essex cream, with and without propylene glycol (PG). The Essex creams that were used in these experiments were without pH adjustment.

### DETAILED DESCRIPTION OF THE INVENTION

Compounds for use according to the invention are defined by general Formula 1 depicted below.

Dermatological disorders can be described as inflammatory and non-inflammatory.

Examples of inflammatory dermatological disorders where compounds of the invention can be used include, but are not limited to, atopic dermatitis, contact dermatitis, psoriasis, acne, rosacea, and seborrheic eczema.

Examples of non-inflammatory dermatological disorders where compounds of the invention can be used include, but are not limited to, melasma, sunburn, benign and malignant skin tumours.

According to the present invention it has been found that compounds of the invention have higher affinity for the kringle 1 motif in plasminogen than EACA and TXA. This should translate into increased clinical efficacy against dermatological disorders compared to EACA and TXA.

Many of the compounds of the invention are more lipophilic than EACA and TXA and this can improve skin penetration.

More potent inhibitors of the plasminogen/plasmin system could be very beneficial and result in improved therapeutic effects, as it may be possible to reach fully effective concentrations in the skin. As can be seen from Table 1 the affinity of compounds of the patent application WO2010117323 (A1) have a higher binding affinity to the isolated kringle 1 compared to EACA and TXA.

Also, EACA and TXA are very hydrophilic compounds resulting in poor skin penetration. In Table 1 are included theoretical calculations of lipophilicity according to ACD log D (pH 7.4), a commonly used fragment based method (http://www.acdlabs.com/resources/freeware/chemsketch/loqp/), for examples of compounds of the present invention that are more lipophilic than TXA and EACA. For hydrophilic and zwitter-ionic compounds it is difficult to obtain comparative experimentally measured log D values, thus calculated values are well suited to compare relative lipophilities between compounds.

Compounds of the invention may have reduced local side effects compared to TXA and EACA as the effective concentrations in the skin can be lower.

For the treatment in humans the compounds of the invention are admixed with a dermatologically acceptable carrier, and subsequently administered topically to the skin. Any suitable, conventional, dermatologically acceptable carrier may be employed. Also, dermatological disorders in animals may be treated with compounds of the invention.

According to a first aspect of the invention, there is provided a compound of Formula 1 or a pharmaceutically acceptable salt, hydrate, solvate or prodrug thereof, for use in the treatment of a dermatological disorder.

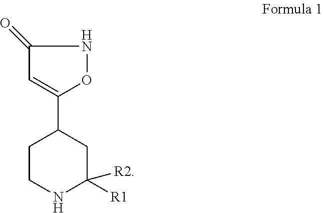

R1 and R2 independently are hydrogen, deuterium, aryl, hetero aryl, C1-C8 alkyl, optionally being substituted with one or more substituents independently being R3,

R3 is an aryl, hetero aryl, fluorine(s), a C1-C6 alkyl containing one or more fluorine, a C1-C6 alkyl containing one or more deuterium, a C1-C6 alkyl containing hydroxy, the aryl and heteroaryl optionally being substituted with one or more halogen, a fluorinated alkoxy, a fluorinated alkyl, a sulfonyl, one or more deuterium, a C1-C6 alkyl, a C1-C6 alkoxy, a nitrile,

or R3 is a C1-C6 alkyl optionally substituted with one or more of the following groups: COOR4, OCOR4, CONR5R6, NR5COR6, OR4;

wherein, R4 is a C1-C10 alkyl optionally substituted with one or more fluorine, deuterium, alkoxy, arylcarboxylate, alkyl carboxylate;

R5 and R6 are independently selected from hydrogen, alkyl or they may together form a 4-8 membered carbon ring;

or R1 and R2 form a 3-10 membered carbon ring optionally comprising O or N and optionally substituted with a C1-C10 alkyl or aryl, hetero aryl optionally substituted with R3.

Pharmaceutically acceptable salts may be both inorganic and organic, one example being a HCl salt.

According to a second aspect of the invention there is provided a compound of Formula 1 or a pharmaceutically acceptable salt, hydrate or solvate thereof, characterized in that the calculated lipophilicity ACD log D (pH 7.4) is greater than −2, for use in the treatment of a dermatological disorder.

According to a third aspect of the invention there is provided a compound, for use in the treatment of dermatological disorders, selected from
5-[(2S,4R)-2-benzylpiperidin-4-yl]-2,3-dihydro-1,2-oxazol-3-one,
5-[(2S,4S)-2-benzylpiperidin-4-yl]-2,3-dihydro-1,2-oxazol-3-one,
5-[(2S,4S)-2-(2-methylpropyl) piperidin-4-yl]-2,3-dihydro-1,2-oxazol-3-one,
5-[(2R,4S)-2-(2-methylpropyl)piperidin-4-yl]-2,3-dihydro-1,2-oxazol-3-one,
5-[(2R,4R)-2-benzylpiperidin-4-yl]-2,3-dihydro-1,2-oxazol-3-one,
5-[(2R,4S)-2-(2,4,5-trifluorophenyl)piperidin-4-yl]-2,3-dihydro-1,2-oxazol-3-one,
5-[(2R,4S)-2-{[3-(trifluoromethyl)-5H,6H,7H,8H-[1,2,4]triazolo[4,3-a]pyrazin-7-yl]methyl}piperidin-4-yl]-2,3-dihydro-1,2-oxazol-3-one,
5-[(2R,4S)-2-{[3-fluoro-4-(trifluoromethyl)phenyl]methyl}piperidin-4-yl]-2,3-dihydro-1,2-oxazol-3-one,
5-[(2R,4S)-2-[(3,5-di-tert-butylphenyl)methyl]piperidin-4-yl]-2,3-dihydro-1,2-oxazol-3-one,
5-[(2R,4S)-2-[(2,4-difluorophenyl)methyl]piperidin-4-yl]-2,3-dihydro-1,2-oxazol-3-one,
5-[(2R,4S)-2-[(3,4-difluorophenyl)methyl]piperidin-4-yl]-2,3-dihydro-1,2-oxazol-3-one,
5-[(2R,4S)-2-[4-(trifluoromethyl)phenyl]piperidin-4-yl]-2,3-dihydro-1,2-oxazol-3-one,
5-[(2S,4S)-2-[(4-tert-butylphenyl)methyl]piperidin-4-yl]-2,3-dihydro-1,2-oxazol-3-one,
5-[(2R,4S)-2-[(4-tert-butylphenyl)methyl]piperidin-4-yl]-2,3-dihydro-1,2-oxazol-3-one,
5-[(2R,4S)-2-[2-fluoro-4-(trifluoromethyl)phenyl]piperidin-4-yl]-2,3-dihydro-1,2-oxazol-3-one,
5-[(2R,4S)-2-(2,4-difluorophenyl)piperidin-4-yl]-2,3-dihydro-1,2-oxazol-3-one,
5-[(2R,4S)-2-(3-tert-butylphenyl)piperidin-4-yl]-2,3-dihydro-1,2-oxazol-3-one,
5-[(2R,4S)-2-[3-methyl-4-(trifluoromethyl)phenyl]piperidin-4-yl]-2,3-dihydro-1,2-oxazol-3-one,
5-[(2R,4S)-2-[6-(trifluoromethyl)pyridin-3-yl]piperidin-4-yl]-2,3-dihydro-1,2-oxazol-3-one,
5-[(2R,4S)-2-[3-fluoro-4-(trifluoromethyl)phenyl]piperidin-4-yl]-2,3-dihydro-1,2-oxazol-3-one,
5-[(2R,4S)-2-(4-fluorophenyl)piperidin-4-yl]-2,3-dihydro-1,2-oxazol-3-one,
5-[(2R,4S)-2-(4-chlorophenyl)piperidin-4-yl]-2,3-dihydro-1,2-oxazol-3-one,
5-[(2R,4S)-2-[(cyclohexyloxy)methyl]piperidin-4-yl]-2,3-dihydro-1,2-oxazol-3-one,
5-[(2R,4S)-2-[2-methyl-4-(trifluoromethyl)phenyl]piperidin-4-yl]-2,3-dihydro-1,2-oxazol-3-one,
5-[(2R,4S)-2-(2-methyl-2H-1,2,3,4-tetrazol-5-yl)piperidin-4-yl]-2,3-dihydro-1,2-oxazol-3-one,
5-[(2R,4S)-2-[2-fluoro-4-(trifluoromethoxy)phenyl]piperidin-4-yl]-2,3-dihydro-1,2-oxazol-3-one,
5-[(2R,4S)-2-{[(2S)-2-(trifluoromethyl)pyrrolidin-1-yl]methyl}piperidin-4-yl]-2,3-dihydro-1,2-oxazol-3-one,
5-[(2R,4S)-2-(4-methanesulfonylphenyl)piperidin-4-yl]-2,3-dihydro-1,2-oxazol-3-one,
5-[(2R,4S)-2-(2,4-dichlorophenyl)piperidin-4-yl]-2,3-dihydro-1,2-oxazol-3-one,
5-[(2R,4S)-2-[(4-methanesulfonylphenyl)methyl]piperidin-4-yl]-2,3-dihydro-1,2-oxazol-3-one,
5-[(2R,4S)-2-[(3,4,5-trifluorophenyl)methyl]piperidin-4-yl]-2,3-dihydro-1,2-oxazol-3-one,
5-[(2R,4S)-2-(2-phenylethyl)piperidin-4-yl]-2,3-dihydro-1,2-oxazol-3-one,
5-[(2S,4S)-2-(2-phenylethyl)piperidin-4-yl]-2,3-dihydro-1,2-oxazol-3-one,
5-[(2R,4S)-2-(2,2-dimethylpropyl)piperidin-4-yl]-2,3-dihydro-1,2-oxazol-3-one,
5-[(2S,4S)-2-(2,2-di methyl propyl)piperidin-4-yl]-2,3-dihydro-1,2-oxazol-3-one
   and
5-[(2R,4S)-2-benzylpiperidin-4-yl]-2,3-dihydro-1,2-oxazol-3-one

In one embodiment of the invention the compound is 5-[(2R,4S)-2-(2,2-dimethylpropyl)piperidin-4-yl]-2,3-dihydro-1,2-oxazol-3-one, or a pharmaceutically acceptable salt, hydrate or solvate thereof, for use in the treatment of a dermatological disorder.

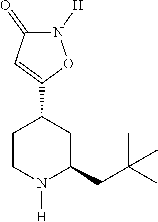 [SMILES: CC(C)(C)C[C@@H]1C[C@@H](c2cc(=O)[nH]o2)CCN1]

### 5-[(2R,4S)-2-(2,2-dimethylpropyl)piperidin-4-yl]-2,3-dihydro-1,2-oxazol-3-one

According to a further aspect of the invention there is provided a pharmaceutical composition, wherein a compound of Formula 1, or a pharmaceutically acceptable salt, hydrate or solvate thereof, is admixed with a dermatologically acceptable carrier, which can be administered topically to skin.

According to another aspect of the invention the compounds as defined herein are used for the manufacturing of a medicament for the treatment of a dermatological disorder as defined herein.

There is also provided a method of treatment of a dermatological disorder, by topical administration of a therapeutically effective amount of a compound of Formula 1 to a mammal, e.g. a human, in need of such treatment.

Compounds of the invention may optionally be formulated together with one or more other therapeutic agents, said agents then being present in therapeutically active amounts. Thus, compound of the invention, or a pharmaceutically acceptable salt, hydrate or solvate thereof, may for example be combined with other drugs that are used or investigated for the treatment of dermatological disorders, including but not limited to azelaic acid, metronidazole, brimonidine, oxymetazoline, omiganan, sulphur, tetracyclines like doxocycline, other antibiotics like erythromycin and sulfacetamide, peroxides like benzoyl peroxide and hydrogen peroxide, ivermectin and similar antiparasitic compounds.

In a pharmaceutical composition incorporating a compound of Formula 1, or a pharmaceutically acceptable salt, hydrate or solvate thereof, the amount of compound of the invention is in the range of about 0.1-5 wt. % of the formulation, more preferably 0.2-2 wt. %.

Pharmaceutical compositions of the invention can be in many forms including, but not limited to, liquids, solutions, lotions, creams, pastes, emulsions, gels, soap bars, sprays, aerosols, micro-emulsions, micro-particles or vesicular dispersions of ionic and/or non-ionic, or wax/aqueous phase dispersions. These compositions are prepared according to standard methods. Such compositions may be applied manually, or using various application devices.

The pharmaceutical compositions of the invention may also comprise any additive commonly used in the dermatological field, compatible with the compounds of the invention. Such additives may be chelating agents, antioxidants, sunscreens, preserving agents, fillers, electrolytes, humectants, colourings, customary bases or acids (inorganic or organic), fragrances, essential oils, active cosmetics, moisturizers, vitamins, essential fatty acids, sphingolipids, self-tanning compounds and agents for soothing and protecting the skin, penetrating agents and gelling agents, or a mixture thereof. These additives and their concentrations are such that they do not harm the advantageous properties of the mixture according to the invention. The additives may be present in the composition in an amount of 0 to 30% by weight relative to the total weight of the composition.

Examples of preservatives include, without limiting them hereto, chlorocresol, phenoxyethanol, benzyl alcohol, diazolidinylurea, parabens, and mixtures thereof.

Examples of humectants include, without limiting them hereto, glycerol, sorbitol, urea propylene glycol, and mixtures thereof.

Examples of chelating agents include, without limiting them hereto, ethylenediamine-tetraacetic acid (EDTA) and its derivatives or its salts, dihydroxyethylglycine, citric acid, tartaric acid and mixtures thereof.

Examples of penetrating agents include, without limiting them hereto, propylene glycol, dipropylene glycol, propylene glycol dipelargonate, lauroglycol, ethoxydiglycol, and mixtures thereof.

When the composition is in emulsion form, the proportion of the oily phase of the emulsion may range, for example from 5 to 80% by weight, and preferably 5 to 50% by weight relative to the total weight of the composition. The oils and emulsifiers used in the composition in emulsion form are chosen from those conventionally used in the dermatological field. The emulsifiers are generally present in the composition in a proportion ranging from 0.3 to 30% by weight, and preferably from 0.5 to 20% by weight relative to the total weight of the composition. The emulsion may also contain lipid vesicles.

Examples of fats include, without limiting them hereto, mineral oils, oils of plant origin, oils of animal origin, synthetic oils, silicone oils, and fluorinated oils. As fatty substances can also be used fatty alcohols such as cetyl alcohol, fatty acids, waxes and gums.

The emulsifier may be anionic, cationic, amphoteric and/or non-ionic.

The anionic and cationic emulsifiers useful in the compositions of this invention should, in addition to the charged group, contain lipophilic groups having from about 6 to about 22 carbon atoms.

Anionic groups are for example carboxylates, sulfonates, phosphonates and the like. Cationic emulsifiers useful in the compositions of this invention include amine salts and/or quaternary ammonium compounds. Amphoteric emulsifiers may include both a basic and an acidic group, e.g. NH₃⁺, —COO—.

Non-ionic surfactants based upon polyethylene glycol ethers of lauryl, cetyl, stearyl and/or oleyl alcohols are useful as emulsifiers in the compositions of this invention. Other useful non-ionic surfactants include fatty acid esters of polyols such as glyceryl stearate, sorbitan tristearate and the oxyethylenated sorbitan stearates (e.g. Tween 60 and Tween 20).

A very useful non-ionic emulsifier for the invention is Cetomacrogol 1000, an emulsifier which is generically known as polyoxyethylene-20 cetyl ether, in the amount of from about 0.5 to about 5 weight percent.

The compositions of this invention may also contain thickening agents, which may be natural thickeners or derivatives thereof. For example, alginates, pectins or carboxy methylcellulose and other cellulosic ethers may be utilized. Other thickening agents that can be included in the compositions of this invention are synthetic thickeners, such as polyacryl and polymethacryl compounds, polyvinylic polymers, polycarboxylic acids and polyethers. Also inorganic thickeners may be used, such as dispersed silica, polysilicates and clay minerals such as montmorillonite, zeolite and phyllosilicates. The thickening agent as described above may be used at a concentration ranging from 0 to 15% and preferably from 0.1 to 5%.

In one embodiment of the invention Essex Cream (Schering AG, Germany) is used. The preservative chlorochresol in this cream may be substituted with benzyl alcohol. Optionally, if pH of the resulting formulation is >6 it may be adjusted to be between 5 and 6.

In another embodiment of the invention Essex Cream to which has been added propylene glycol and/or glycerol is used, the combined concentrations of propylene glycol and glycerol ranging from 0-20%, preferably 5-15%. The preservative chlorochresol in this cream may be substituted with benzyl alcohol. Optionally, if pH of the resulting formulation is >6 it may be adjusted to be between 5 and 6.

Topical administration of the formulation may be 1-4 times daily, preferably 1-2 times daily.

### EXAMPLES

The following examples illustrate the invention without limiting it hereto.

### Example 1

Binding affinities to plasminogen kringle 1 for the compounds of the present invention were measured by NMR as described below:

The NMR experiment identifies compounds binding to the lysine binding pocket in a competition assay where tranexamic acid was used as reference compound and 1D T1rho experiments were used for detection. Signal intensities from free tranexamic acid are reduced when recombinant kringle 1 protein is added due to binding to this protein. The NMR signals from tranexamic acid regain intensity when an added compound displaces it from the lysine binding pocket of kringle 1.

The compounds were run one-by-one at a concentration between 15 and 300 μM. The concentration of protein and tranexamic acid were 10 and 100 μM respectively. A test compound resulting in at least 10% increase in tranexamic acid intensity (displacement) was considered binding to kringle 1. Relative Kd values were calculated using a Kd value of 1 μM for tranexamic acid.

A 1D T1rho experiment was used for the measurements and was performed at 293K. Spectra were recorded with a spin-lock time of 200 ms, 256 scans and a relaxation delay of 2 s.

Table 1 shows binding affinities to plasminogen kringle 1 for compounds of the present invention compared to tranexamic acid (TXA) epsilon amino-caproic acid (EACA). Also, calculated lipophilicity values are included (ACD log D, pH 7.4).

TABLE 1

| | | Calculated |
|---|---|---|
| | Kringle 1 | ACD LogD |
| NAME | Kd μM | (7.4) |
| 5-[(2S,4R)-2-benzylpiperidin-4-yl]-2,3- | 0.5 | −0.22 |
| dihydro-1,2-oxazol-3-one | | |
| 5-[(2S,4S)-2-benzylpiperidin-4-yl]-2,3- | 0.2 | −0.22 |
| dihydro-1,2-oxazol-3-one | | |
| 5-[(2S,4S)-2-(2-methylpropyl)piperidin-4- | 0.2 | −0.54 |
| yl]-2,3-dihydro-1,2-oxazol-3-one | | |
| 5-[(2R,4S)-2-(2-methylpropyl)piperidin-4- | 0.2 | −0.54 |
| yl]-2,3-dihydro-1,2-oxazol-3-one | | |
| 5-[(2R,4R)-2-benzylpiperidin-4-yl]-2,3- | 0.1 | −0.22 |
| dihydro-1,2-oxazol-3-one | | |
| 5-[(2R,4S)-2-(2,4,5-trifluorophenyl)piperidin- | 0.3 | 1.8 |
| 4-yl]-2,3-dihydro-1,2-oxazol-3-one | | |
| 5-[(2R,4S)-2-{[3-(trifluoromethyl)- | 0.24 | −0.33 |
| 5H,6H,7H,8H-[1,2,4]triazolo[4,3- | | |
| a]pyrazin-7-yl]methyl}piperidin- | | |
| 4-yl]-2,3-dihydro-1,2-oxazol-3-one | | |
| 5-[(2R,4S)-2-{[3-fluoro-4- | 0.24 | 1.41 |
| (trifluoromethyl)phenyl]methyl}piperidin- | | |
| 4-yl]-2,3-dihydro-1,2-oxazol-3-one | | |
| 5-[(2R,4S)-2-[(3,5-di-tert- | 1 | 3.24 |
| butylphenyl)methyl]piperidin- | | |
| 4-yl]-2,3-dihydro-1,2-oxazol-3-one | | |
| 5-[(2R,4S)-2-[(2,4- | 0.1 | 0.46 |
| difluorophenyl)methyl]piperidin- | | |
| 4-yl]-2,3-dihydro-1,2-oxazol-3-one | | |
| 5-[(2R,4S)-2-[(3,4- | 0.1 | 0.46 |
| difluorophenyl)methyl]piperidin- | | |
| 4-yl]-2,3-dihydro-1,2-oxazol-3-one | | |
| 5-[(2R,4S)-2-[4- | 0.1 | 1.81 |
| (trifluoromethyl)phenyl]piperidin-4- | | |
| yl]-2,3-dihydro-1,2-oxazol-3-one | | |
| 5-[(2S,4S)-2-[(4-tert- | 0.1 | 1.72 |
| butylphenyl)methyl]piperidin- | | |
| 4-yl]-2,3-dihydro-1,2-oxazol-3-one | | |
| 5-[(2R,4S)-2-[(4-tert- | 0.3 | 1.72 |
| butylphenyl)methyl]piperidin- | | |
| 4-yl]-2,3-dihydro-1,2-oxazol-3-one | | |
| 5-[(2R,4S)-2-[2-fluoro-4- | 0.3 | 2.28 |
| (trifluoromethyl)phenyl]piperidin-4-yl]- | | |
| 2,3-dihydro-1,2-oxazol-3-one | | |
| 5-[(2R,4S)-2-(2,4-difluorophenyl)piperidin- | 0.2 | 1.31 |
| 4-yl]-2,3-dihydro-1,2-oxazol-3-one | | |
| 5-[(2R,4S)-2-(3-tert-butylphenyl)piperidin- | 0.28 | 1.65 |
| 4-yl]-2,3-dihydro-1,2-oxazol-3-one | | |
| 5-[(2R,4S)-2-[3-methyl-4- | 0.1 | 2.25 |
| (trifluoromethyl)phenyl]piperidin-4-yl]- | | |
| 2,3-dihydro-1,2-oxazol-3-one | | |
| 5-[(2R,4S)-2-[6- | 0.28 | 0.42 |
| (trifluoromethyl)pyridin-3-yl]piperidin-4- | | |
| yl]-2,3-dihydro-1,2-oxazol-3-one | | |
| 5-[(2R,4S)-2-[3-fluoro-4- | 0.2 | 2.08 |
| (trifluoromethyl)phenyl]piperidin-4-yl]- | | |
| 2,3-dihydro-1,2-oxazol-3-one | | |
| 5-[(2R,4S)-2-(4-fluorophenyl)piperidin-4- | 0.6 | 0.8 |
| yl]-2,3-dihydro-1,2-oxazol-3-one | | |
| 5-[(2R,4S)-2-(4-chlorophenyl)piperidin-4- | 0.1 | 1.44 |
| yl]-2,3-dihydro-1,2-oxazol-3-one | | |
| 5-[(2R,4S)-2- | 0.3 | 0.38 |
| [(cyclohexyloxy)methyl]piperidin-4- | | |
| yl]-2,3-dihydro-1,2-oxazol-3-one | | |
| 5-[(2R,4S)-2-[2-methyl-4- | 0.3 | 2.17 |
| (trifluoromethyl)phenyl]piperidin-4-yl]- | | |
| 2,3-dihydro-1,2-oxazol-3-one | | |
| 5-[(2R,4S)-2-(2-methyl-2H-1,2,3,4- | 0.2 | −0.02 |
| tetrazol-5-yl)piperidin-4-yl]-2,3- | | |
| dihydro-1,2-oxazol-3-one | | |
| 5-[(2R,4S)-2-[2-fluoro-4- | 0.3 | 2.1 |
| (trifluoromethoxy)phenyl]piperidin-4- | | |
| yl]-2,3-dihydro-1,2-oxazol-3-one | | |
| 5-[(2R,4S)-2-{[(2S)-2- | 0.4 | −1.07 |
| (trifluoromethyl)pyrrolidin-1- | | |
| yl]methyl}piperidin-4-yl]- | | |
| 2,3-dihydro-1,2-oxazol-3-one | | |
| 5-[(2R,4S)-2-(4- | 0.2 | 0.49 |
| methanesulfonylphenyl)piperidin-4- | | |
| yl]-2,3-dihydro-1,2-oxazol-3-one | | |
| 5-[(2R,4S)-2-(2,4-dichlorophenyl)piperidin- | 0.2 | 2.5 |
| 4-yl]-2,3-dihydro-1,2-oxazol-3-one | | |
| 5-[(2R,4S)-2-[(4- | 0.4 | −0.85 |
| methanesulfonylphenyl)methyl]piperidin- | | |
| 4-yl]-2,3-dihydro-1,2-oxazol-3-one | | |
| 5-[(2R,4S)-2-[(3,4,5- | 0.14 | 0.56 |
| trifluorophenyl)methyl]piperidin-4- | | |
| yl]-2,3-dihydro-1,2-oxazol-3-one | | |
| 5-[(2R,4S)-2-(2-phenylethyl)piperidin- | 0.1 | 0.42 |
| 4-yl]-2,3-dihydro-1,2-oxazol-3-one | | |
| 5-[(2S,4S)-2-(2-phenylethyl)piperidin- | 0.1 | 0.42 |
| 4-yl]-2,3-dihydro-1,2-oxazol-3-one | | |
| 5-[(2R,4S)-2-(2,2-dimethylpropyl)piperidin- | 0.1 | −0.11 |
| 4-yl]-2,3-dihydro-1,2-oxazol-3-one | | |
| 5-[(2S,4S)-2-(2,2-dimethylpropyl)piperidin- | 0.1 | −0.11 |
| 4-yl]-2,3-dihydro-1,2-oxazol-3-one | | |
| 5-[(2R,4S)-2-benzylpiperidin-4-yl]-2,3- | 0.3 | −0.22 |
| dihydro-1,2-oxazol-3-one | | |
| 4-(aminomethyl)cyclohexane-1-carboxylic | 1 | −2.00 |
| acid (TXA) | | |
| 6-aminohexanoic acid (EACA) | 31.62 | −2.27 |

### Example 2

Inhibition of Fibrinolysis in Human Plasma Clot-Lysis Assay (McCormack 2012)

This example illustrates the higher potency of 5-[(2R,4S)-2-(2,2-dimethylpropyl)piperidin-4-yl]-2,3-dihydro-1,2-oxazol-3-one in comparison with tranexamic acid (TXA) and epsilon amino-caproic acid (EACA).

Inhibition of fibrinolysis was measured in an assay system using citrated platelet poor plasma by addition of tPA and CaCl2. The formation of fibrin is started when the citrated plasma is re-calcified which leads to endogenous formation of thrombin. The fragmentation of fibrin particles is then initiated by tPA activation of plasminogen to plasmin. Inhibition of fibrinolysis results in longer lifetime of the fibrin clot. Blood from healthy fat-fasting volunteers was collected into 0.109 M trisodium citrate (9 to 1), the tubes were centrifuged at 2000×g, for 20 min, at room temperature and the supernatant (the platelet poor plasma) was pooled, aliquoted and frozen at −85° C. Recombinant human tPA (Altplase, Actilyse, Boehringer Ingelheim) was used. At the day of experiment the plasma was thawed and all constituents, except tPA, were pre-warmed to 37° C. To each a half-volume microtiter plate well was added 9 μl compound solution or saline, 10 μl CaCl2 (final concentration 7.5 mM) and 20 μl saline. Five parts ice-cold tPA (final conc 80 ng/ml), was mixed with 45 parts plasma immediately before adding 50 μl of this mixture to each well. The plates were read in a SpectraMax® Reader at 37° C. and 405 nm with a reading interval of 2 min. Data were collected for 20 hrs. Fibrin formation and dissolution were measured as the time period between half amplitude at clot formation (t1) and at 15% clot dissolution (t2), both calculated from the difference between maximal absorbance and absorbance prior to start of plasma coagulation. The effect on fibrinolysis was calculated as percent of no inhibition according to the equation:

%=100*((t2−t1)vehicle/(t2−t1)cmpd).

The IC50 and IC90 were defined as the clot-lysis time being prolonged to twice and nine times the vehicle value and was calculated with the software GraFit 32.

5-[(2R,4S)-2-(2,2-dimethylpropyl)piperidin-4-yl]-2,3-dihydro-1,2-oxazol-3-one, had an IC50 of 0.94 μM compared to tranexamic acid (TXA) which had an IC50 6.66 μM and epsilon amino-caproic acid (EACA) which had an IC50 72.7 μM. However, the therapeutic plasma concentration of TXA in the treatment of bleeding disorders is in the range of 30-90 μM (McCormack 2012), which corresponds to roughly 90% inhibition (IC90) of fibrinolysis in the clot-lysis assay for tranexamic acid (TXA). The IC90 of 5-[(2R,4S)-2-(2,2-dimethylpropyl)piperidin-4-yl]-2,3-dihydro-1,2-oxazol-3-one was 3.26 μM compared to the IC90 of tranexamic acid (TXA) of 55.4 μM and epsilon amino-caproic acid (EACA) of 637 μM.

### Example 3

50 mg of 5-[(2R,4S)-2-(2,2-dimethylpropyl)piperidin-4-yl]-2,3-dihydro-1,2-oxazol-3-one was formulated in 10 g of ESSEX cream. Addition of 5-[(2R,4S)-2-(2,2-dimethylpropyl)piperidin-4-yl]-2,3-dihydro-1,2-oxazol-3-one to Essex cream increases the pH of the cream roughly 1 pH unit as measured using pH indicator sticks (Acilit, Merck, KGaA, Darmstadt, Germany), by first wetting the sticks with distilled water and then spreading a thin layer of the Essex formulation over the indicators. To compensate for the pH increase the half molar amount of concentrated phosphoric acid (relative to the molar amount of 5-[(2R,4S)-2-(2,2-dimethylpropyl)piperidin-4-yl]-2,3-dihydro-1,2-oxazol-3-one) was added to the Essex cream. The restitution of the pH decreased the long term crystal formation in the formulation as measured with a Zeiss Axioskop with camera Infinity 2 using software Infinity Analyze release 6.3.0 (Carl Zeiss Microscopy, LLC, United States).
60 g of Essex cream (Schering AG, Germany) contain:
9 g vaselinum album
3.6 g paraffinum liquidum
4.32 g cetostrearyl alcohol
1.35 g cetomacrogol 1000
0.06 g chlorocresol
0.18 g sodium dihydrogen phosphate dehydrate
0.0012 g concentrated phosphoric acid (85%)
41.488 g distilled water

### Example 4

70 mg of 5-[(2R,4S)-2-(2,2-dimethylpropyl)piperidin-4-yl]-2,3-dihydro-1,2-oxazol-3-one was formulated in 10 g of ESSEX B cream with 20% propylene glycol (Apotek Produktion & Laboratorier AB (APL)). Addition of 5-[(2R,4S)-2-(2,2-dimethylpropyl)piperidin-4-yl]-2,3-dihydro-1,2-oxazol-3-one to Essex B cream increases the pH of the cream roughly 1 pH unit as measured using pH indicator sticks (Acilit, Merck, KGaA, Darmstadt, Germany), by first wetting the sticks with distilled water and then spreading a thin layer of the Essex formulation over the indicators. To compensate for the pH increase the half molar amount of concentrated phosphoric acid (relative to the molar amount of 5-[(2R,4S)-2-(2,2-dimethylpropyl)piperidin-4-yl]-2,3-dihydro-1,2-oxazol-3-one) was added to the Essex cream. The restitution of the pH decreased the long term crystal formation in the formulation as measured with a Zeiss Axioskop with camera Infinity 2 using software Infinity Analyze release 6.3.0 (Carl Zeiss Microscopy, LLC, United States). 100 g cream ESSEX B cream with 20% propylene glycol (Apotek Produktion & Laboratorier AB (APL)) contain:
Propylene glycol 20 g
cetomakrogol 1000
cetostearyl alcohol
liquid paraffin
white vaselin
sodium di-hydrogen phosphate di-hydrate
conc. phosphoric acid
water
benzyl alcohol 1%

### Example 5

70 mg of 5-[(2R,4S)-2-(2,2-dimethylpropyl)piperidin-4-yl]-2,3-dihydro-1,2-oxazol-3-one was formulated in 10 g of ESSEX Cream to which had been added 5% propylene glycol and 5% glycerol. Addition of 5-[(2R,4S)-2-(2,2-dimethylpropyl)piperidin-4-yl]-2,3-dihydro-1,2-oxazol-3-one to Essex cream increases the pH of the cream roughly 1 pH unit as measured using pH indicator sticks (Acilit, Merck, KGaA, Darmstadt, Germany), by first wetting the sticks with distilled water and then spreading a thin layer of the Essex formulation over the indicators. To compensate for the pH increase the half molar amount of concentrated phosphoric acid (relative to the molar amount of 5-[(2R,4S)-2-(2,2-dimethylpropyl)piperidin-4-yl]-2,3-dihydro-1,2-oxazol-3-one) was added to the Essex cream. The restitution of the pH decreased the long term crystal formation in the formulation as measured with a Zeiss Axioskop with camera Infinity 2 using software Infinity Analyze release 6.3.0 (Carl Zeiss Microscopy, LLC, United States).

60 g of Essex Cream (Schering AG, Germany), before adding propylene glycol and glycerol, contain:
9 g vaselinum album
3.6 g paraffinum liquidum
4.32 g cetostrearyl alcohol
1.35 g cetomacrogol 1000
0.06 g chlorocresol
0.18 g sodium dihydrogen phosphate dehydrate
0.0012 g concentrated phosphoric acid (85%)
41.488 g distilled water

### Example 6

Penetration of 5-[(2R,4S)-2-(2,2-dimethylpropyl)piperidin-4-yl]-2,3-dihydro-1,2-oxazol-3-one through porcine skin membranes in vitro. Pig ear skin was used in this work, as it represents a relevant model to human skin in terms of anatomy, lipid composition, permeability, and electrical properties (Bjorklund 2013).

To investigate the effect of varying the concentration of 5-[(2R,4S)-2-(2,2-dimethylpropyl)piperidin-4-yl]-2,3-dihydro-1,2-oxazol-3-one in phosphate buffered saline (PBS) solutions and in Essex cream, both with/without 20% propylene glycol (PG), a modified Franz cell equipment was used. Penetration of the compound through skin membranes was determined using flow-through diffusion cells mounted in a heat block to keep a constant temperature of 32° C. Receptor solution of PBS (pH 7.4) was continuously pumped through the cells with a flow rate of 1.5 ml/h and collected in vials at defined time intervals (2 h). Before applying the test solution in infinite doses, the membranes were hydrated by letting the receptor solution flow through the receptor chamber for 1 hour. The experiment was initiated when ˜2 ml of test formulation was applied into the donor chamber (effective diffusion area 0.64 cm²). The donor chamber was sealed with parafilm during the experiment.

Pig ears were obtained fresh from a local abattoir and frozen at −80° C. until use. Split-thickness skin membranes (approx. 500 μm thick) were prepared from tissue of the inside of the outer ear by using a dermatome. Circular membranes (16 mm in diameter) were cut out to fit the diffusion cells (9 mm in diameter).

In the calculation of steady state flux, five time points between 16 and 24 h was used for skin membranes.

An HPLC assay was used for analysis of 5-[(2R,4S)-2-(2,2-dimethylpropyl)piperidin-4-yl]-2,3-dihydro-1,2-oxazol-3-one. The mobile phase was a mixture of acetonitrile and 10 mM ammonium acetate. The pre-column guard filter was Column Saver, 2 μm, (IT Inc) and the column was Waters XBridge™ C18 3.5 μm, 3.0×50 mm. The column temperature was ambient, the flow rate was 0.65 mL/min and wavelength was 220 nm. Injection volume was 10 μL and run time 5 min.

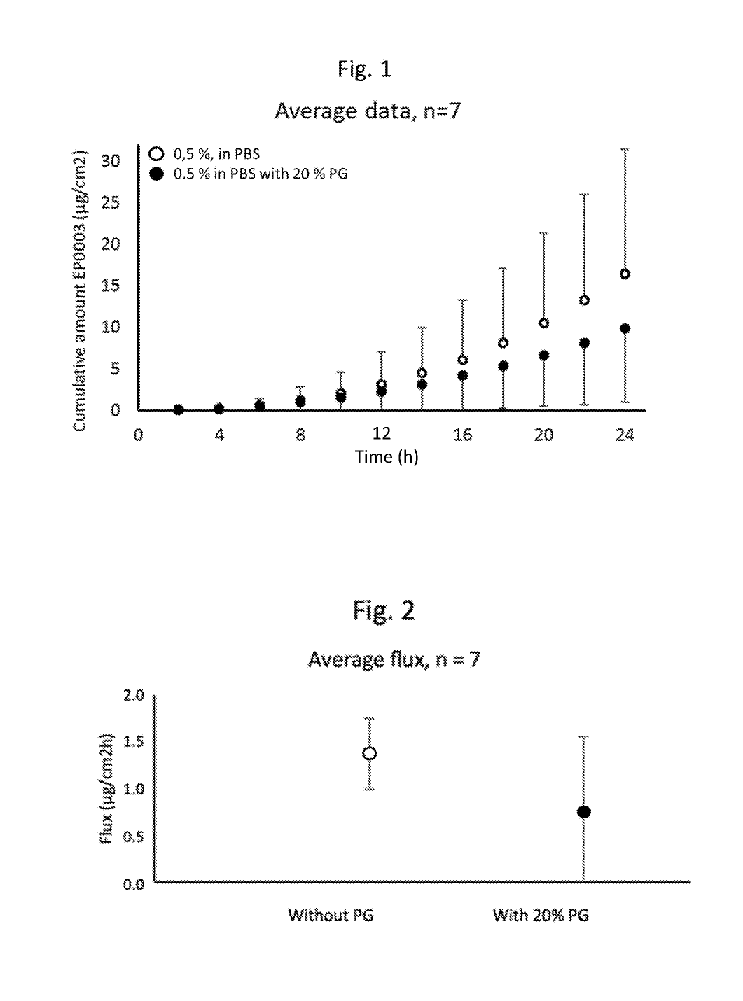

The obtained data shows that 5-[(2R,4S)-2-(2,2-dimethylpropyl)piperidin-4-yl]-2,3-dihydro-1,2-oxazol-3-one penetrates the pig ear in formulations both with and without propylene glycol (PG) as illustrated in FIG. 1-3.

FIG. 1 is a graph showing the cumulative amount on the receiver side of the pig skin membrane as a function of time, of 0.5% 5-[(2R,4S)-2-(2,2-dimethylpropyl)piperidin-4-yl]-2,3-dihydro-1,2-oxazol-3-one, dissolved in phosphate buffered saline (PBS), with and without propylene glycol (PG).

FIG. 2 is a graph showing the steady state flux through the pig skin membrane of 0.5% of 5-[(2R,4S)-2-(2,2-dimethylpropyl)piperidin-4-yl]-2,3-dihydro-1,2-oxazol-3-one, dissolved in phosphate buffered saline (PBS), with and without propylene glycol (PG).

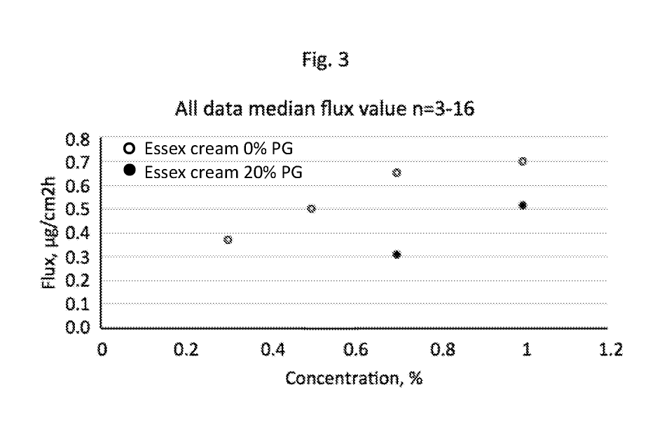

FIG. 3 is a graph showing the steady state flux through the pig skin membrane of various concentrations of 5-[(2R,4S)-2-(2,2-dimethylpropyl)piperidin-4-yl]-2,3-dihydro-1,2-oxazol-3-one dissolved in Essex cream, with and without propylene glycol (PG). The Essex creams that were used in these experiments were without pH adjustment.

### REFERENCES, PATENT APPLICATIONS

WO2010117323 (A1)

### REFERENCES, OTHER SOURCES

Ayuthaya P K N, Niumphradit N, Manosroi A, Nakakes A. Topical 5% tranexamic acid for the treatment of melasma in Asians: A double-blind randomized controlled clinical trial. J Cosmet Laser Therapy 2012; 3:150-4.
Bjorklund S. Skin hydration—How water and osmolytes influence biophysical properties of stratum corneum. Thesis 2013, ISBN 978-91-7422-325-5.
Ceruti P, Principe M, Capello M, Capello P, Novelli F. Three are better than one: plasminogen receptors as cancer theranostic targets. Experiment hematol Oncol 2013; 2:12, 11 pages.
CYCLO-f, EMA 27 Jul. 2000 (http://www.ema.europa.eu/docs/en_GB/document_library/Referrals_document/Cyklo_f_29/WC500011080.pdf)
Denda M, Kitamura K, Elias P M, Feingold K R. trans-4-(Aminomethyl)cyclohexane carboxylic acid (T-AMCHA), and anti-fibrinolytic agent, accelerates barrier recovery and prevents the epidermal hyperplacia induced by epidermal injury in hairless mice and humans. J Invest Dermatol 1997; 109:84-90.
Duffy M J, McGowan P M, Harbeck N, Thomssen C, Manfred Schmitt M. uPA and PAI-1 as biomarkers in breast cancer: validated for clinical use in level-of-evidence-1 studies. Breast Cancer Research 2014, 16:428
Dunn C J, Goa K L. Tranexamic acid. A review of its use in surgery and other indications. Drugs 1999; 57:1005-32.
Kitamura K, Yamada K, I to A, Fukuda M. Research on the mechanism by which dry skin occurs and the development of an effective compound for its treatment. J Soc Cosmet Chem 1995; 29:133-45.
Jensen P J, Baird J, Morioka S, Lessin S, Lazarus G S. Epidermal plasminogen activator in abnormal cutaneous lesions. J Invest Dermatol 1988; 90:777-82.
Jensen P J, Baird J, Belin D, Vassalli J D, Busso N, Gubler P, Lazarus G S. Tissue plasminogen activatior in psoriasis. J Invest Dermatol 1990; 95:13S-14S.
Kim M S, Chang S U, Haw S, Bak H, Kim Y J, Lee M W. Tranexamic acid solution soakings an excellent approach for rosacea patients: a preliminary observation in six patients. J Dermatol 2012; 40:70-1
Li Q, Ke F, Zhang W, Shen X, Xu Q, Wang H, Yu X Z, Leng Q, Wang H. Plasmin Plays an Essential Role in Amplification of Psoriasiform Skin Inflammation in Mice. PLoS ONE 2011; 6:e16483. doi:10.1371/journal.pone.0016483
Longhurst H, Cicardi M. Hereditary angio-oedema. Lancet 2012; 379:474-81.
Madureira P A, O'Connell P A, Surette A P, Miller V A, Waisman D M. The Biochemistry and Regulation of S100A10: A multifunctional plasminogen receptor involved in oncogenesis. J Biomed Biotechnol 2012; Article ID 353687, 21 pages.
McCormack P L. Tranexamic acid. A review of its use in the treatment of hyperfibrinolysis. Drugs 2012; 72:585-617
Plow E F, Doeuvre L, Das R. So many plasminogen receptors: why? J Biomed Biotechnol 2012 Article ID 141806, 6 pages.
Rawlings A V, Voegeli R. Stratum corneum proteases and dry skin conditions. Cell Tissue Res 2013; 351:217-35.
Spiers E M, Lazarus G S, Lyons-Giordano B. Expression of plasminogen activator enzymes in psoriatric epidermis. J Invest Dermatol 1994; 102:333-8.
Sun N, Niu Y, Chen C, Zhong S, Liu H, Wu Y. The influence of tranexamic acid on skin barrier function and inflammation in rosacea. J Clin Dermatol 2013; 42:345-7.
Syrovets T, Lunov O, Simmet T. Plasmin as a proinflammatory cell activator. J Leukocyte Biol 2012; 92:509-19
Tse T W, Hui E. Tranexamic acid: an important adjuvant in the treatment of melasma. J Cosmet Dermatol 2013; 12, 57-66.
Two Am, Hata T R, Nakatsuji T, Coda A B, Kotol P F, Wu W, Shafiq F, Huang E Y, Gallo R L. J investing Dermatol 2013; doi:10.1028/jid.2013.472.
Wu Y, Chen C, Zhong S, Niu Y, Liu H. The influence of skin barrier function and LL-37 in rosacea. J Dermatol 2010; 37(Suppl. 1):p 76, abstract 90413.
Zhong S, Sun N, Liu H, Niu Y, Chen C, Wu Y*. Topical tranexamic acid improves the permeability barrier in rosacea Dermatologica Sinica Volume 33, Issue 2, June 2015, Pages 112-117
Yuan C, Wang X M, Yang L J, Wu P L. Tranexamic acid accelerates skin barrier recovery and upregulates occludin in damaged skin. Internat J Dermatol 2014; 53:959-65.

1. A compound of Formula 1, 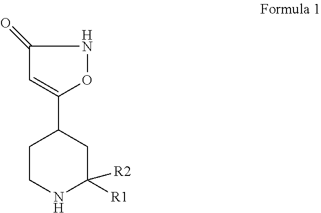
wherein
R1 and R2 are each selected from the group consisting of hydrogen, deuterium, aryl, hetero aryl, C1-C8 alkyl, optionally being substituted with one or more substituents independently being R3, and a 3-10 membered ring formed by R1 and R2 optionally comprising a selected element selected from the group consisting of O and N and optionally substituted with a C1-C10 alkyl or aryl, hetero aryl optional substituted with R3;
R3 is selected from the group consisting of an aryl, hetero aryl, fluorine(s), a C1-C6 alkyl containing one or more fluorines, a C1-C6 alkyl containing one or more deuterium, a C1-C6 alkyl containing hydroxy, the aryl and heteroaryl optionally being substituted with one or more halogen, a fluorinated alkoxy, a fluorinated alkyl, a sulfonyl, one or more deuterium, a C1-C6 alkyl, a C1-C6 alkoxy, a nitrile, and a C1-C6 alkyl optionally substituted with one or more groups selected from the group consisting of COOR4, OCOR4, CONR5R6, NR5COR6 and OR4;
wherein R4 is a C1-C10 alkyl optionally substituted with one or more fluorine, deuterium, alkoxy, arylcarboxylate, alkyl carboxylate;
R5 and R6 are selected from the group consisting of hydrogen, alkyl and a 4-8 membered carbon ring formed by R5 and R6;
and including pharmaceutically suitable salts, hydrates or solvates thereof, for use in the treatment of a dermatological disorder.

2. The compound according to claim 1, characterized in that the calculated lipophilicity ACD log D (pH 7.4) is greater than −2.

3. The compound according to claim 1, wherein the compound is selected from
5-[(2S,4R)-2-benzylpiperidin-4-yl]-2,3-dihydro-1,2-oxazol-3-one,
5-[(2S,4S)-2-benzylpiperidin-4-yl]-2,3-dihydro-1,2-oxazol-3-one,
5-[(2S,4S)-2-(2-methylpropyl)piperidin-4-yl]-2,3-dihydro-1,2-oxazol-3-one,
5-[(2R,4S)-2-(2-methylpropyl)piperidin-4-yl]-2,3-dihydro-1,2-oxazol-3-one,
5-[(2R,4R)-2-benzylpiperidin-4-yl]-2,3-dihydro-1,2-oxazol-3-one,
5-[(2R,4S)-2-(2,4,5-trifluorophenyl)piperidin-4-yl]-2,3-dihydro-1,2-oxazol-3-one,
5-[(2R,4S)-2-{[3-(trifluoromethyl)-5H,6H,7H,8H-[1,2,4]triazolo[4,3-a]pyrazin-7-yl]methyl}piperidin-4-yl]-2,3-dihydro-1,2-oxazol-3-one,
5-[(2R,4S)-2-{[3-fluoro-4-(trifluoromethyl)phenyl]methyl}piperidin-4-yl]-2,3-dihydro-1,2-oxazol-3-one,
5-[(2R,4S)-2-[(3,5-di-tert-butylphenyl)methyl]piperidin-4-yl]-2,3-dihydro-1,2-oxazol-3-one,
5-[(2R,4S)-2-[(2,4-difluorophenyl)methyl]piperidin-4-yl]-2,3-dihydro-1,2-oxazol-3-one,
5-[(2R,4S)-2-[(3,4-difluorophenyl)methyl]piperidin-4-yl]-2,3-dihydro-1,2-oxazol-3-one,
5-[(2R,4S)-2-[4-(trifluoromethyl)phenyl]piperidin-4-yl]-2,3-dihydro-1,2-oxazol-3-one,
5-[(2S,4S)-2-[(4-tert-butylphenyl)methyl]piperidin-4-yl]-2,3-dihydro-1,2-oxazol-3-one,
5-[(2R,4S)-2-[(4-tert-butylphenyl)methyl]piperidin-4-yl]-2,3-dihydro-1,2-oxazol-3-one,
5-[(2R,4S)-2-[2-fluoro-4-(trifluoromethyl)phenyl]piperidin-4-yl]-2,3-dihydro-1,2-oxazol-3-one,
5-[(2R,4S)-2-(2,4-difluorophenyl)piperidin-4-yl]-2,3-dihydro-1,2-oxazol-3-one,
5-[(2R,4S)-2-(3-tert-butylphenyl)piperidin-4-yl]-2,3-dihydro-1,2-oxazol-3-one,
5-[(2R,4S)-2-[3-methyl-4-(trifluoromethyl)phenyl]piperidin-4-yl]-2,3-dihydro-1,2-oxazol-3-one,
5-[(2R,4S)-2-[6-(trifluoromethyl)pyridin-3-yl]piperidin-4-yl]-2,3-dihydro-1,2-oxazol-3-one,
5-[(2R,4S)-2-[3-fluoro-4-(trifluoromethyl)phenyl]piperidin-4-yl]-2,3-dihydro-1,2-oxazol-3-one,
5-[(2R,4S)-2-(4-fluorophenyl)piperidin-4-yl]-2,3-dihydro-1,2-oxazol-3-one,
5-[(2R,4S)-2-(4-chlorophenyl)piperidin-4-yl]-2,3-dihydro-1,2-oxazol-3-one,
5-[(2R,4S)-2-[(cyclohexyloxy)methyl]piperidin-4-yl]-2,3-dihydro-1,2-oxazol-3-one,
5-[(2R,4S)-2-[2-methyl-4-(trifluoromethyl)phenyl]piperidin-4-yl]-2,3-dihydro-1,2-oxazol-3-one,
5-[(2R,4S)-2-(2-methyl-2H-1,2,3,4-tetrazol-5-yl)piperidin-4-yl]-2,3-dihydro-1,2-oxazol-3-one,
5-[(2R,4S)-2-[2-fluoro-4-(trifluoromethoxy)phenyl]piperidin-4-yl]-2,3-dihydro-1,2-oxazol-3-one,
5-[(2R,4S)-2-{[(2S)-2-(trifluoromethyl)pyrrolidin-1-yl]methyl}piperidin-4-yl]-2,3-dihydro-1,2-oxazol-3-one,
5-[(2R,4S)-2-(4-methanesulfonylphenyl)piperidin-4-yl]-2,3-dihydro-1,2-oxazol-3-one,
5-[(2R,4S)-2-(2,4-dichlorophenyl)piperidin-4-yl]-2,3-dihydro-1,2-oxazol-3-one,
5-[(2R,4S)-2-[(4-methanesulfonylphenyl)methyl]piperidin-4-yl]-2,3-dihydro-1,2-oxazol-3-one,
5-[(2R,4S)-2-[(3,4,5-trifluorophenyl)methyl]piperidin-4-yl]-2,3-dihydro-1,2-oxazol-3-one,
5-[(2R,4S)-2-(2-phenylethyl)piperidin-4-yl]-2,3-dihydro-1,2-oxazol-3-one,
5-[(2S,4S)-2-(2-phenylethyl)piperidin-4-yl]-2,3-dihydro-1,2-oxazol-3-one,
5-[(2R,4S)-2-(2,2-dimethylpropyl)piperidin-4-yl]-2,3-dihydro-1,2-oxazol-3-one,
5-[(2S,4S)-2-(2,2-dimethylpropyl)piperidin-4-yl]-2,3-dihydro-1,2-oxazol-3-one and
5-[(2R,4S)-2-benzylpiperidin-4-yl]-2,3-dihydro-1,2-oxazol-3-one.

4. The compound according to claim 1, wherein the compound is 5-[(2R,4S)-2-(2,2-dimethylpropyl)piperidin-4-yl]-2,3-dihydro-1,2-oxazol-3-one.

5. The compound-according to claim 3, wherein the disorder is an inflammatory dermatological disorder.

6. The compound according to claim 5, wherein the inflammatory dermatological disorder is selected from the group consisting of atopic dermatitis, contact dermatitis, psoriasis, acne, rosacea, and seborrheic eczema.

7. The compound according to claim 3, wherein the disorder is a non-inflammatory dermatological disorder.

8. The compound according to claim 7, wherein the non-inflammatory dermatological disorder is selected from the group consisting of melasma, sunburn, benign and malignant skin tumours.

9. The compound according to claim 3, wherein the dermatological disorder is rosacea.

10. The compound according to claim 3, wherein the dermatological disorder is melisma.

11. The compound according to claim 3, wherein the dermatological disorder is psoriasis.

12. A pharmaceutical composition, comprising a compound according to claim 3 admixed with a dermatologically acceptable carrier for topical administration to skin.

13. The pharmaceutical composition according to claim 12 characterized in that it is an oil in water emulsion.

14. The pharmaceutical composition according to claim 12 characterized in that the dermatologically acceptable carrier is composed of vaselinum album, paraffinum liquidum, cetostrearyl alcohol, cetomacrogol 1000, chlorocresol or bensylalcohol, sodium dihydrogen phosphate dehydrate, concentrated phosphoric acid (85%) and distilled water.

15. The pharmaceutical composition according to claim 12 characterized in that the dermatologically acceptable carrier is composed of propylene glycol and/or glycerol, vaselinum album, paraffinum liquidum, cetostrearyl alcohol, cetomacrogol 1000, chlorocresol or bensylalcohol, sodium dihydrogen phosphate dehydrate, concentrated phosphoric acid (85%) and distilled water.

16. The compound according to claim 3 in combination with azelaic acid, metronidazole, brimonidine, oxymetazoline, omiganan, sulphur, tetracyclines like doxocycline, erythromycin, sulfacetamide, peroxides like benzoyl peroxide and hydrogen peroxide, ivermectin and similar antiparasitic compounds, for the treatment and prophylaxis of dermatological disorders.
